(19) United States
(12) Patent Application Publication
(10) Pub. No.: US 2020/0276007 A1
(43) Pub. Date: Sep. 3, 2020

(54) MULTILAYERED BIOLOGIC MESH AND METHODS OF USE THEREOF

(71) Applicant: Mubayiwa Cornelious MUSARA, Fulton, MD (US)

(72) Inventor: Mubayiwa Cornelious MUSARA, Fulton, MD (US)

(21) Appl. No.: 16/875,370

(22) Filed: May 15, 2020

Related U.S. Application Data
(63) Continuation of application No. PCT/US2018/061498, filed on Nov. 16, 2018.
(60) Provisional application No. 62/587,754, filed on Nov. 17, 2017.

(51) Int. Cl. A61F 2/00; A61N 5/10; A61L 27/36

(52) U.S. Cl. CPC A61F 2/0063; A61N 5/1001; A61L 27/3604; A61F 2210/0004; A61F 2210/0076; A61F 2210/0095; A61F 2002/0081; A61N 2005/1024; A61F 2250/0067

(57) ABSTRACT

Systems and methods for using surgical meshes to deliver chemotherapeutic agents and radioactive elements are presented herein. The surgical mesh may comprise multiple layers, with an inner or outer layer comprising the radioactive element, and an inner or outer layer comprising the chemotherapeutic layer. Upon exposure to physiological conditions, the surgical mesh along with pellets or powdered elements embedded therein biodegrades.

### CROSS-REFERENCE TO RELATED APPLICATIONS

This is a continuation of International Application PCT/US2018/061498, with an international filing date of Nov. 16, 2018, which claims priority to U.S. Provisional Application No. 62/587,754, filed on Nov. 17, 2017, all of which are hereby incorporated by reference in their entirety.

### FIELD OF THE INVENTION

The field of the invention is related to implantable surgical meshes, and in particular, to multilayered biologic meshes for delivery of one or more chemotherapeutic agents and one or more radioactive elements to a patient.

### BACKGROUND OF THE INVENTION

The information provided in the background of the invention is not an admission that any of the information provided herein is prior art or relevant to the claimed subject matter. In the event that a term of an incorporated reference is used in an inconsistent manner as compared to the same term in this application, the definition of the term provided in this application shall apply.

Traditional methods of cancer treatment, including chemotherapy and radiation therapy, have a number of substantial drawbacks. For example, chemotherapeutic agents delivered intravenously circulate throughout the vascular system, and are delivered to both healthy and cancerous tissues. Not only does this method of delivery cause damage to healthy tissues, but also, chemotherapeutic agents delivered intravenously are often unable to reach non-vascular or poorly vascularized diseased tissues, allowing the tumor to persist or metastasize. Accordingly, chemotherapeutic agents may not be delivered at a high enough concentration to be effective, with higher concentrations having detrimental effects.

Radiation therapy, on the other hand, generally delivers radiation in one or more treatments distributed across a period of time (e.g., typically over several weeks to months). However, radiation therapy can damage healthy tissues, and result in unpleasant side effects, e.g. damage to the GI tract resulting in side effects such as nausea, vomiting, and diarrhea and anatomical damage including perforation and bowel obstruction. Other side effects include damage to the heart and lungs, loss of hair, decreased immunity, and subsequent risk of infections, hair loss, nerve damage, pain, etc.

Advances in intraoperative brachytherapy have evolved from external beam radiation to intraoperative implantation of radioactive pellets, which are used to treat a variety of cancers. However, external beam radiation has significant comorbidities, and the implanted radioactive pellets are not biodegradable and are permanent. Although the implanted pellets may not have long term effects when placed in solid tissues, some patients opt for removal of these non-degradable pellets, which requires an undesirable subsequent surgery. Additionally, the non-degradable pellets may be subject to translocation, and the pellets could migrate to a different region of the body and may adversely affect healthy tissue.

While certain types of synthetic meshes, such as synthetic vicryl mesh, have been combined with non-biodegradable radioactive pellets for use in thoracic lung surgery, this approach, too, has additional drawbacks due to high rates of complications from infections, and other adverse reactions from patients, including pellet migration.

Even though various techniques using radiation therapy and chemotherapy are known in the art, all suffer from serious drawbacks. Additionally, cancer cells may evade these modes of delivery, e.g., through inaccessibility or development of resistance (e.g., mutations). Therefore, there is still a need to provide improved compositions and methods to eliminate tumor cells.

### SUMMARY

Present invention embodiments are directed towards a multilayered biologic mesh comprising one or more radioactive elements and one or more chemotherapeutic agents embedded therein.

In some embodiments, the mesh is formed from porcine tissue (e.g., porcine urinary bladder) or human tissue or any other suitable tissue derived from a mammal. In other embodiments, the mesh is formed from biosynthetic materials. In still other embodiments, the mesh is formed from any combination of porcine, human, or biosynthetic material. A variety of suitable materials are known in the art, and all such materials are specifically contemplated for use herein (see, e.g., Gupta et al., Hernia (2006) v10: 419-425).

In some embodiments, the mesh is bioabsorbable. The bioabsorbable mesh acts as a carrier biologic matrix to deliver a combination of radioactive elements and chemotherapeutic agents to a particular site in the body. The bioabsorbable mesh becomes incorporated into an area of the body through incorporation. The rate of biodegradation and incorporation can be determined and controlled using the manufacturing process to control mesh characteristics (e.g., number of layers, porosity of layers, thickness of layers, composition of layers, etc.), and in some cases, the rate of biodegradation and incorporation may be designed to range from days to months. As the bioabsorbable mesh undergoes absorption, radioactive elements and chemotherapeutic agents are released at the site of implantation, and are eventually excreted from the body based on the element utilized. By delivering this therapeutic treatment, which includes one or more radioactive elements and one or more chemotherapeutic agents, using a bioabsorbable mesh, treatment is directed to the site of the tumor, while minimizing the effects of radiation and chemotherapy to local healthy tissue e.g., by asymmetrical thickness and composition of the mesh, wherein one side is engineered to be less permeable and/or to degrade over a longer time period. As a further benefit of this approach, pellets and radioactive elements embedded within the biological mesh do not become displaced. Thus, the mesh allows for higher, continuous doses of radiation and chemotherapy to be delivered to a particular tumor site at sequential or concurrent times, with less injury and fewer residual effects to the healthy surrounding tissue.

In still other embodiments, the multilayered mesh may comprise layers of different materials such that the outer layers are bioabsorbed more quickly than the inner layers. Such hybrid multilayered meshes may deliver chemotherapeutic agents according to different time frames as compared to radioactive elements. For example, the radioactive elements embedded or dispersed throughout the outer layer provide continuous radiation therapy over an initial period of time.

The chemotherapeutic agents embedded or dispersed throughout the inner layers of the mesh may bioabsorb slowly, delivering a concentrated dose of chemotherapy over a longer timeframe, after the radiation has dispersed.

In still other embodiments, the mesh may be designed to release a first dose of chemotherapy, concurrent with or prior to radiation release. Once the release of radiation is concluded or nearly concluded, the mesh may be designed to release a second dose of chemotherapy on a longer time frame, such that the patient receives: a first quick initial dose of chemotherapy embedded in one or more outer layers, a dosage of radiation embedded in dissolvable pellets in one or more inner layers, and a second dose of chemotherapy embedded within inner layers that release the chemotherapeutic on a slower timeframe than dissolution of the pellets.

According to the techniques disclosed herein, the one or more radioactive elements and the one or more chemotherapeutic agents may be dispersed throughout the layers of the mesh or may be provided in the form of pellets embedded in the multi-layered mesh. Pellets may be formed using a dissolvable polymer (e.g., polymers derived from natural sources such as collagen, polysaccharides, microbial polyesters, etc; synthetic degradable polymers such as aliphatic polyesters including polyglycolic acid, polylactic acid, polycaprolactone and polydioxanone; polyortho esters; polyanhydrides; degradable polycarbonates; polyamino acids (in which conventional peptide bonds have been modified or replaced with other linkages; etc.) mixed with radioactive elements and/or chemotherapeutic agents. Dissolvable materials are known in the art, and all such materials are contemplated for use herein (e.g., Pulapura et al., J. of Biomaterials Applications, (1992) v:6, p 216-250).

As the absorbable mesh becomes incorporated or remodeled into the recipient tissue, the pellet (including radioactive elements, chemotherapeutic agents, or both) releases chemotherapeutic agents and/or radioactive elements at the site of implantation, which is eventually excreted by the patient. Thus, heavy metals used for radiation treatment are not retained within the recipient, and therefore, there is no waste or byproducts from this method. For example, if the radioactive element is iodine, then iodine would be excreted from the body via urine. Thus, it is contemplated that the pellets described herein are fully biodegradable, and that the therapeutic within the pellet is excreted from the body as the pellet biodegrades.

In some embodiments, the multilayered mesh may comprises pores. For example, the size of the pores may range from about 75 um (e.g., to allow infiltration by various red and white blood cells) to more than several millimeters (e.g., from 3-5 mm). In other embodiments, pores may increase the available surface area of the mesh to promote more rapid release of chemotherapeutic agents or radioactive elements. In still other embodiments, pores may help reduce unwanted side effects from biological responses to implanted meshes, e.g., the occurrence of foreign body reactions (e.g., granuloma formation), as well as reduce associated infections. In other embodiments, the mesh may not contain pores of an appreciable size.

In some embodiments, the multilayered mesh comprises chemotherapeutic agents dispersed or embedded in the outer layers and radioactive elements embedded in the inner layers. In still other embodiments, the multilayered mesh comprises both chemotherapeutic agents and radioactive elements dispersed or embedded throughout the various layers.

In still other embodiments, the chemotherapeutic agent(s) may be embedded within one or more layers of the mesh in the form of a pellet, e.g., as a pellet in the outer layers of the multilayered mesh, as a pellet in the inner layer(s) of the mesh; as a pellet throughout all layers of the multilayered mesh.

In other embodiments, the radioactive element(s) may be embedded within one or more layers of the mesh in the form of a pellet, e.g., as a pellet in the outer layers of the multilayered mesh, as a pellet in the inner layer(s) of the mesh; as a pellet throughout all layers of the multilayered mesh.

In some embodiments, the size of the pellets ranges from 100 μm up to 5 mm.

In still other embodiments, one or more layers of the mesh may be immersed in a solution comprising either the chemotherapeutic agent, the radioactive element, or both, allowing the therapeutic to become dispersed throughout the mesh. In still other embodiments, each layer of the mesh may be coated with a solution comprising either the chemotherapeutic agent, the radioactive element, or both. In still other embodiments, each layer of the mesh may be formed using 3D printing technology using a biological solution comprising either the chemotherapeutic agent, the radioactive element, or both and in addition to polymers or other biological materials (e.g., collagen, fibronectin, laminin, proteoglycans, etc.) to form the scaffold of the mesh.

For pellets containing both chemotherapeutic agents and radioactive elements, the pellet is configured such that the chemotherapeutic agent is released from the pellet at a rate that locally enhances or augments the effect of the radiation. The duration of the release of chemotherapeutic agent(s) may be designed to match the duration of the radiation. In some embodiments, the combination of the chemotherapeutic agent and radioactive element may have improved efficacy or synergy over a simple additive effect of the chemotherapeutic agent and the radioactive element.

In some embodiments, the radioactive pellets are designed to emit a predetermined dose of radioactivity with an associated penetration (e.g., the distance that the radiation can penetrate into surrounding tissue), which may be pre-calculated based upon cancer type, location, and stage.

In still other embodiments, the rate of absorption or biodegradation of the biologic mesh may be designed to parallel the rate of absorption or biodegradation of the pellets, such that the biological mesh and the pellets biodegrade within similar time frames.

In some embodiments, the mesh is formed using high compression or bonding technology. In this approach, individual layers of the mesh comprise one or more chemotherapeutic agents and/or one or more radioactive elements, which may be dispersed throughout the mesh or embedded as pellets. The layers are stacked on top of each other, and compression or bonding are used to join the layers of the mesh together. Thus, the multilayered meshes as described herein may be formed using high compression technology to generate a multilayer mesh composed of any combination of suitable materials, including but not limited to porcine urinary bladder, human tissue, or other biosynthetic material, or any combination thereof.

The mesh is designed for implantation to kill tumor cells. In some embodiments, the mesh is implanted into a tumor or tumor bed to kill off tumors. In other embodiments, the mesh may be implanted in the general area from which the tumor was surgically removed to kill off residual or trace amounts of tumor cells. In other embodiments, the mesh may be implanted in areas in which surgical resection of the tumor is incomplete to reduce the size of or kill off tumor cells.

In some embodiments, the mesh is implanted in areas with poor vascularity, in which traditional methods of delivering chemotherapeutic agents or radioactive elements are not effective or would result in major surgical complications. For example, the meshes described herein are suitable for pelvic neoplasms, as the pelvis has poor vascularity and limited accessibility, e.g., foramen within the pelvis have limited accessibility or are completely inaccessible to traditional modes of delivery.

The surgical meshes or pellets as described herein can be used to treat a variety of different types of cancers, including but not limited to, prostate cancer, liver cancer, or brain cancer. The meshes as described herein are suitable for use in the urogenital region (e.g., to treat prostate, ovarian, cervical, vaginal, uterine, ureter, or bladder cancers or tumors). The meshes as described herein are suitable for use in the treatment of cancers occurring in the abdominal region, e.g., colorectal cancers or tumors, or cancers within abdominal organs. The meshes as described herein are suitable for use in the treatment of intra-peritoneal neoplasms (e.g., within the liver, within the gastric bed, or within the colonic bed). The meshes as described herein are suitable for use in the treatment of thoracic cavity neoplasms (e.g., within the lung, within the mediastinal, or within the pleural regions). In other embodiments, the meshes as described herein are suitable for implantation within the abdominal region to treat retroperitoneal neoplasms. Retroperitoneal neoplasms generally occur outside major organs, and include but are not limited to tumors found in fat, fibrous tissue, peripheral nerve, skeletal muscle, or vessels as well as the abdominal region (e.g., pancreatic bed, kidney bed, or other soft tissues). These tumors include but are not limited to: extra-gonadal germ cell tumours, fibrosarcomas, hemangiopericytomas, leiomyosarcomas, liposarcomas, malignant fibrous histiocytomas, malignant peripheral nerve sheath tumours, primary retroperitoneal adenocarcinomas, rhabdomyosarcomas and metastatic tumours to the retroperitoneum. Many other types of cancers are suitable for treatment according to the methods and compositions disclosed herein, and all such types are contemplated herein.

In other embodiments, the mesh may additionally act as a dividing barrier, e.g., positioned to exclude the bowel from the pelvis. In such embodiments, the mesh may be thicker and may degrade more slowly along the side facing the bowel, and may be thinner and degrade more quickly along the side facing the tumor. Additionally, the portion of the mesh facing the tumor may be enriched in chemotherapeutic agents and radioactive elements, while the side of the mesh facing the bowel (normal tissue) may be depleted in chemotherapeutic agents and radioactive elements, thus directionally releasing therapeutic agents to the tumor site. In still other embodiments, the mesh additionally functions as a filler and enhances regeneration in patients with significant tissue/structural defects while locally delivering therapeutic agents.

The surgical meshes have a variety of advantages over traditional modes of delivering chemotherapy or radiotherapy. These advantages include allowing for local delivery of both radiation and chemotherapy, which together may have additive or synergistic effects. Still another advantage is that the pellets or the mesh with embedded pellets are fully bioabsorbable, and do not require a subsequent operation for removal. Additionally, in some embodiments, the mesh promotes regeneration of damaged tissue with decreased scarring while delivering therapeutic agents. Further, the mesh reduces complication rates from infections and from wound breakdown, as compared to other methods of cancer treatment, such as external beam intraoperative radiation. The multilayered mesh, by targeting cancer cells, especially residual cancer cells, in a continuous manner, can result in a higher cure rate and decreased rates of recurrence as compared to traditional methods of cancer treatment.

Various features, aspects and advantages of the embodiments presented herein will become more apparent from the following detailed description of preferred embodiments.

### Definitions

As used herein, the term “bioabsorbable” refers to a rate of absorption that correlates with biological remodeling and usually occurs over days to months and can be engineered to years if necessary. When meshes are implanted, the nature of the mesh (e.g., composition, porosity, and shape) allows cells from the recipient to enter the mesh and adhere. This results in incorporation and remodeling as there is gradual replacement of mesh by autologous protein with an influx of cells and formation of new vessels concurrent with mesh absorption. These cells can degrade the mesh and regenerate the degraded scaffold by secreting extracellular proteins. Thus, over a period of time, the mesh will be bioabsorbed or undergo changes though the process of remodeling resulting in new functional tissue where there previously were defects.

As used herein, the term “chemotherapeutic agent” refers to a compound, e.g., chemical, biologic/immunologic or hormonal, capable of killing a tumor or cancer cell.

As used herein, the term “radioactive element” includes an element that emits radiation capable of killing a tumor or cancer cell.

As used herein, the term “multilayer mesh” refers to multiple layers of tissue, e.g., of porcine, human, biosynthetic origin or other biodegradable material. The layers may be compressed or bonded together to form a multilayer mesh, or the multilayered mesh may be generated using 3D printing techniques.

As used herein, the term “pellet” refers to a chemotherapeutic agent and/or a radioactive element mixed with a biodegradable polymer in the form of a pellet. Thus, pellets are biodegradable, and may be embedded throughout the mesh. In some cases, the pellets can be designed, e.g., through size or shape or polymer material, to biodegrade or bioabsorb at the same rate as the mesh. In other cases, the pellets can be designed, e.g., through size or shape or polymer material, to biodegrade or bioabsorb at a higher rate than the mesh.

As used herein, the term “patient” includes any living organism, including humans and animals.

As used herein, the term under “physiological conditions” refers to exposure to an aqueous-based solution at relevant physiologic temperature of about 10° C. to about 45° C., preferably between 36-42° C.

As used herein, the term “therapeutic agent” refers to any molecule designed to treat cancer or to aid in the treatment of cancer. In some embodiments, therapeutic agents include but are not limited to antibodies or fragments thereof, antigens (with or without adjuvants), anti-inflammatory agents, anti-neoplastic agents, aptamers, chemotherapeutic agents, hormonal, immunostimulating agents, immunosuppressive agents, lipids, nucleic acid constructs, nucleotides, peptides, polysaccharides, protein scaffolds, RNA, radioactive elements, small molecules, vaccines, vaccines with adjuvants, vectors, viral vectors, viruses (live or inactivated), etc. It is specifically contemplated that the mesh or pellets may be formulated using any suitable biologic, pharmaceutical or nutraceutical for delivery to a subject.

### BRIEF DESCRIPTION OF THE DRAWINGS

FIGS. 1A-ID are illustrations showing layers of an example surgical multilayer mesh, according to embodiments presented herein.

FIGS. 2A-2B are illustrations showing layers of another example surgical multilayer mesh, according to embodiments presented herein.

FIGS. 3A-3C are illustrations showing example surgical multilayer meshes with differing pore sizes or no pores, according to embodiments presented herein.

FIGS. 4A-4D are illustrations showing layers of example pellets that may be embedded in the multilayer mesh, according to embodiments presented herein.

FIGS. 5A-5B are illustrations showing an example mesh with pellets embedded therein, according to embodiments presented herein.

FIGS. 6A-6C are illustrations showing additional examples of meshes with pellets embedded therein, according to embodiments presented herein.

The examples presented herein are not intended to be limiting. It is understood that many different variations of these examples are disclosed within the application, and that all such embodiments fall within the scope of present invention embodiments.

### DETAILED DESCRIPTION

Present invention embodiments are directed towards a multilayered biologic mesh comprising one or more radioactive elements and one or more chemotherapeutic agents embedded therein. It is understood that the meshes may be formed of any number of layers, and the examples presented herein are intended to be non-limiting. For example, an inner layer may comprise one or more layers, though a single layer may be shown in reference to a figure. Similarly, an outer layer may comprise one or more layers, though a single layer may be shown in reference to a figure.

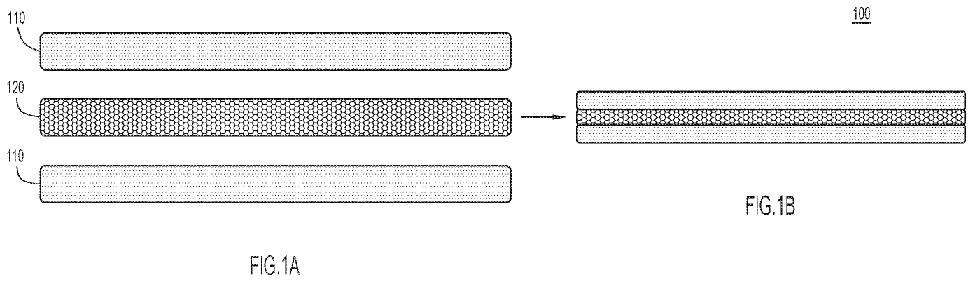

FIGS. 1A-1B are example illustrations of a multilayered mesh 100. In FIG. 1A, three layers of mesh are formed, for example, two outer layers 110 and an inner layer 120. The layers undergo bonding or compression, e.g., using high compression technology, to form a multilayered mesh 100, as shown in FIG. 1B. In this example, the outer layers 110 contain one or more chemotherapeutic agents dispersed throughout, while the inner layers contain a radioactive element dispersed throughout. The thickness and number of layers determine the rate of bioabsorbability.

In this example, the radioactive element may be combined with (e.g., by immersing the layer in a solution comprising the therapeutic, by applying a coating of the therapeutic to the layer, by applying a powder or gel comprising the therapeutic to the layer, etc.) the biodegradable material used to form a layer of the mesh, allowing a homogenous or substantially homogeneous distribution of the radioactive element throughout the mesh. Similarly, the chemotherapeutic agent may be mixed with the biodegradable material used to form a layer of the mesh, allowing a homogenous or substantially homogeneous distribution of the chemotherapeutic agent throughout the mesh.

Manufacturing processes utilizing bonding may use adhesives to join individual layers of the mesh together, while compression may use compressive force to join individual layers of the mesh together.

FIGS. 1C-1D are example illustrations of another multilayered mesh 105. In FIG. 1C, three layers of mesh are formed, for example, two outer layers 120 and an inner layer 110. The layers undergo bonding or compression, e.g., using high compression technology or adhesive forces, to form a multilayered mesh 105, as shown in FIG. 1D. In this example, the outer layers 120 contain one or more radioactive agents or pellets dispersed throughout, while the inner layers contain one or more chemotherapeutic agents dispersed throughout. The thickness and number of layers determine the rate of bioabsorbability.

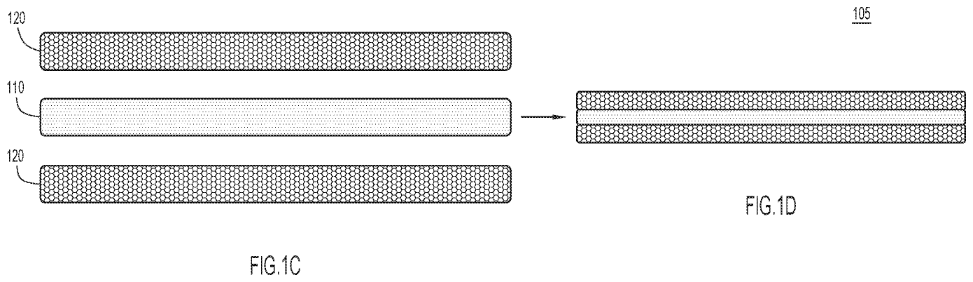

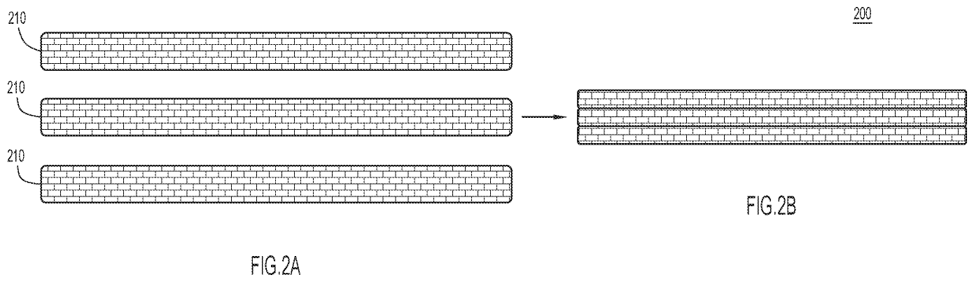

FIGS. 2A-2B are illustrations of another example of a multilayer mesh 200. In FIG. 2A, three layers of mesh 210 are formed. In this example, the three layers of mesh each comprise both one or more chemotherapeutic agents and one or more radioactive elements. The layers may undergo bonding or compression, e.g., using high compression technology, to form multilayered mesh 200, as shown in FIG. 2B. In this example, each layer 210 contains a chemotherapeutic agent dispersed throughout and a radioactive element dispersed throughout.

In this example, the radioactive element and the chemotherapeutic agent may be combined with (e.g., by immersing the layer in a solution comprising the therapeutic, by applying a coating of the therapeutic to the layer, by applying a powder or gel comprising the therapeutic to the layer, etc.) the biodegradable material used to form layers of the mesh, allowing homogenous or substantially homogeneous distribution of the chemotherapeutic agent and the radioactive element throughout the mesh.

In some embodiments, layer 110 comprising one or more chemotherapeutic agents may be combined with layer 210 comprising one or more radioactive agents (dispersed or as pellets) and one or more chemotherapeutic agents. In other embodiments, layer 120 comprising one or more radioactive agents (dispersed or as pellets) may be combined with layer 210 comprising one or more radioactive agents (dispersed or as pellets) and one or more chemotherapeutic agents. In this example, layer 110 may be an inner or an outer layer, layer 120 may be an inner or an outer layer, and layer 210 may be an inner or an outer layer.

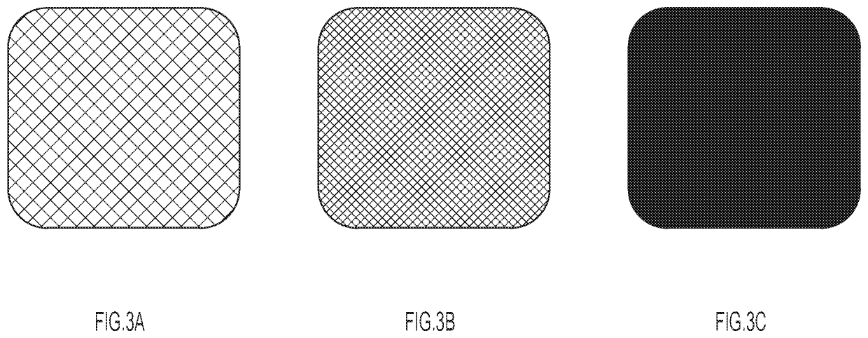

Referring to FIGS. 3A-3C, various illustrations of meshes having differing pore sizes are present. FIG. 3A shows an example mesh comprising pores of a relatively large size, e.g., from 1-5 mm. FIG. 3B shows an example mesh comprising pores of an intermediate size, e.g., from 50 μm-1 mm. FIG. 3C shows an example mesh considered not to be porous.

Pores may be of any suitable size, e.g., from about 50 μm up to 5 mm or any size in between. For example, the diameter of the pore may be 50 μm, 55 μm, 60 μm, 65 μm, 70 μm, 75 μm, 85 μm, 90 μm, 95 μm, 100 μm, 200 μm, 300 μm, 400 μm, 500 μm, 600 μm, 700 μm, 800 μm, 900 μm, 1 mm, 2 mm, 3 mm, 4 mm, 5 mm, and so forth, or any value in between.

In some embodiments, the pore size may help modulate the rate of bioabsorption or biodegradation of the surgical mesh and release of chemotherapeutic agents. For example, increasing the pore size may increase the available surface area of the mesh to promote or increase release of chemotherapeutic agents or radioactive elements. Decreasing the pore size may decrease the available surface area of the mesh to promote slower release of chemotherapeutic agents or radioactive elements.

In still other embodiments, pores may help reduce unwanted side effects from implanted meshes, e.g., the occurrence of foreign body reactions (e.g., granuloma formation), as well as reducing associated infections. In some embodiments, the size of the pores may be about 75 μm (e.g., to allow infiltration by various red and white blood cells and rapid neovascularization).

In general, the surgical mesh may be of any suitable shape or size, e.g., square, rectangular, oval, circular, diamond, etc. The shape and size of the surgical mesh may be determined by the target location of the mesh, e.g., prostate, liver, intestines, etc. The multilayered mesh may be cut to any suitable geometry, governed by the particular location, type of cancer, dimensions of patient, etc. to provide a suitable, customized fit. Once implanted, the meshes may be surgically glued or sutured in place. The shape of the mesh and the positioning of the therapeutic within the mesh depends upon the application. Accordingly, the mesh can be specifically configured and customized to deliver therapeutics to a specific target region.

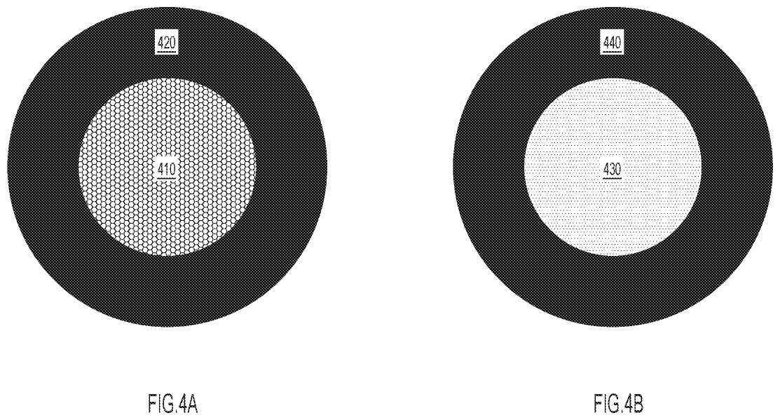

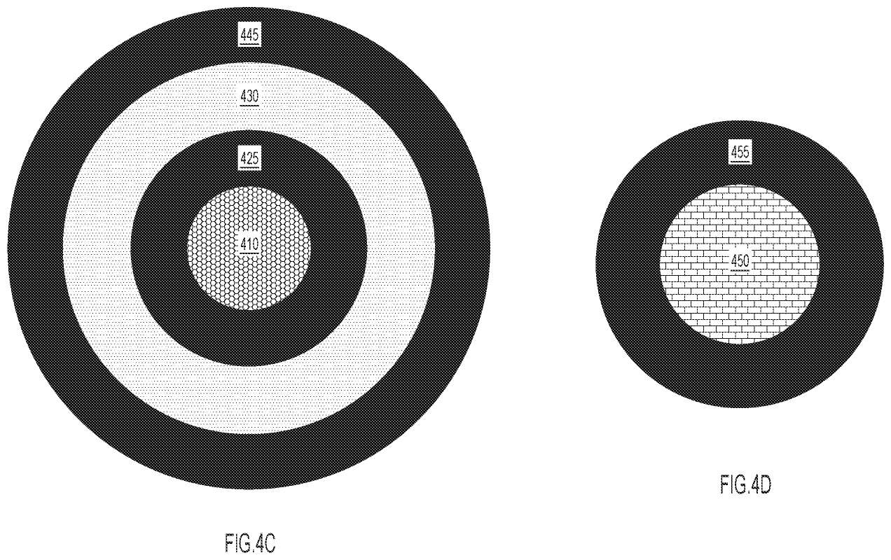

FIGS. 4A-4D show various illustrations of a biodegradable pellet. In some embodiments, the pellet may be embedded within a mesh. In other embodiments, the pellet may be administered directly (not within a mesh). Pellets may be distributed throughout each layer of a multilayered mesh, or in other embodiments, may be distributed throughout a subset of layers of a multilayered mesh.

Pellets may range in size from 100 μm up to 5 mm, or any size in between. The pellets may be uniformly shaped or irregularly shaped. In some embodiments, radioactive pellets may range in size from about 0.002 μm to about 200 μm, from 10 μm to 100 μm, and from about 20-50 μm.

The pellet can take on a variety of shapes. In other embodiments, the pellet may take the shape of a sphere. In still other embodiments, the pellet may take the shape of a grain of rice, or cylinder. Here, it is understood that many different type of geometries are suitable and all are within the scope of the embodiments described herein. For the following figures, it is understood that the pellet comprises one or more biodegradable polymers mixed with one or more chemotherapeutic agents and one or more radioactive elements.

FIG. 4A shows an illustration of an example pellet, according to the embodiments set forth herein. In this example, the inner layer 410 comprises a radioactive element. An optional outer layer 420, may act as a coating, e.g., to help control the rate of biodegradation of the pellet.

In some embodiments, pellets may be formed using radioactive powders. Radioactive powders include but are not limited to I¹²⁵, Pd¹⁰³, Rn⁸⁶, Rn²²², Y⁹⁰, P³² or Au¹⁹⁸. In some embodiments, a single type of radioactive power is mixed with a polymer to form a pellet, while in other embodiments; multiple types of radioactive powders are mixed with a polymer to form a pellet. The radiation pellet produces a field that is uniform or substantially uniform in all directions.

For applications in which a higher radiation penetration is optimal, particles that emit gamma radiation may be selected, e.g., Rn²²² or Au¹⁹⁸. For applications, in which a lower radiation penetration is optimal, particles that emit beta particles may be selected.

Pellets may be placed in the tumor bed or within the tissue to be treated (interstitial therapy), or within a region from which the tumor has been removed (intracavitary therapy).

FIG. 4B shows an illustration of an example pellet, according to the embodiments set forth herein. In this example, the inner layer 430 comprises a chemotherapeutic agent. An optional outer layer 440, may act as a coating, e.g., to help control the rate of biodegradation of the pellet. Outer layer 440 may be the same or different from layer 420.

FIG. 4C shows an illustration of an example pellet, according to the embodiments set forth herein. In this example, the inner layer 410 comprises a radioactive element. An optional middle layer 425, may act as a coating, e.g., to separate the chemotherapeutic layer 430 from the radioactive layer 410. Layer 430 comprises a chemotherapeutic agent. An optional outer layer 445, may act as a coating, e.g., to help control the rate of biodegradation of the pellet. Outer layer 440 may the same or different from layer 420 or 440. In this example, the pellet may be fully biodegradable.

FIG. 4D shows an illustration of an example pellet, according to the embodiments set forth herein. In this example, the inner layer 450 comprises a blend of chemotherapeutic agent(s) and radioactive element(s). An optional outer layer 455, may act as a coating, e.g., to help control the rate of dissolution of the pellet. Outer layer 455 may be the same or different from layer 445, 420, or 440.

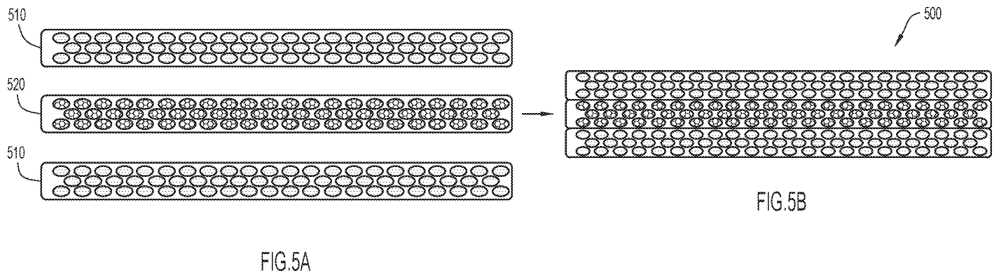

Turning now to FIGS. 5A-5B, example illustrations of a multilayered mesh 500 are presented with pellets dispersed throughout. In FIG. 5A, three layers of mesh are formed, for example, two outer layers 510 and an inner layer 520. The layers undergo bonding or compression, e.g., using high compression technology, to form a multilayered mesh 500, as shown in FIG. 5B. In this example, the outer layers 510 contain a chemotherapeutic agent in the form of a pellet, wherein the pellets are dispersed throughout the outer layer, while the inner layer contains a radioactive element in the form of a pellet dispersed throughout the inner layer 520.

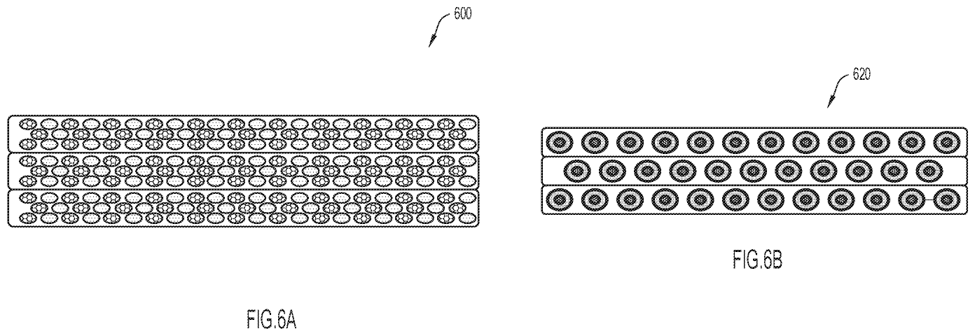

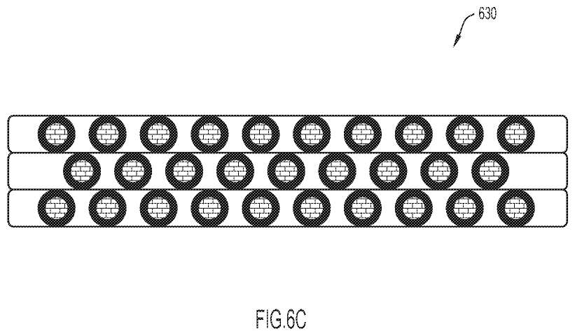

Turning now to FIGS. 6A-6C, example illustrations of a multilayered mesh 600 are presented with pellets dispersed throughout. The meshes are bioabsorbed as a function of time. Three layers of mesh are formed, for example, two outer layers and an inner layer, and undergo bonding or compression, e.g., using high compression technology, to form multilayered mesh 600, as shown in FIG. 6A. In this example, each layer contains a chemotherapeutic agent in the form of a pellet (see, FIG. 4B) and a radioactive element in the form of a pellet (see, FIG. 4A) dispersed throughout each layer.

As shown in FIG. 6B, three layers of mesh are formed, for example, two outer layers and an inner layer, and undergo bonding or compression, e.g., using high compression technology, to form a multilayered mesh 620. In this example, each layer has embedded pellets, the pellets each containing a chemotherapeutic agent and a radioactive element (See, FIG. 4C) dispersed throughout each layer.

As shown in FIG. 6C, three layers of mesh are formed, for example, two outer layers and an inner layer, that undergo bonding or compression, e.g., using high compression technology, to form a multilayered mesh 630, as shown in FIG. 6C. In this example, each layer contains a chemotherapeutic agent and a radioactive element in the same pellet (See, FIG. 4D) dispersed throughout each layer.

The amount of radioactive element required to provide therapeutic levels of radiation varies depending upon: (1) the particular radioisotope or combination of radioisotopes, (2) preparation of the isotope, and (3) the particular therapeutic application. In some embodiments, 0.1-5 millicuries, or 1-2 millicuries, per centimeter is a suitable target for radiation delivery. It is presumed that the mechanical integrity of the biodegradable polymer will not be impaired by the dosage of radioactivity delivered. In some embodiments, the particles are layered uniformly over the inner layers of mesh.

In some embodiments, the pellets containing the radioactive element may be spaced 0.1 cm, 0.2 cm, 0.3 cm, 0.4 cm, 0.5 cm apart, 1 cm apart, 2 cm apart, 3 cm apart, 4 cm apart, 5 cm apart, or more, or any unit in between.

In some embodiments, particles released into the body from biodegradation/bioabsorption of the mesh or pellet, include biocompatible particles such as palladium particles that are not known to be toxic when implanted interstitially, or yttrium oxide particles also are not associated with toxic effects.

Suitable biodegradable polymers include but are not limited to: polymers derived from natural sources such as collagen, polysaccharides, microbial polyesters, etc; synthetic degradable polymers such as aliphatic polyesters including polyglycolic acid, polylactic acid, polycaprolactone and polydioxanone; polyortho esters; polyanhydrides; degradable polycarbonates; mono and poly amino acids in which conventional peptide bonds have been modified or replaced with other linkages; etc. and mixed with radioactive elements and/or chemotherapeutic agents. Dissolvable materials are known in the art, and all such materials are contemplated for use herein (e.g., Pulapura et al., J. of Biomaterials Applications, (1992) v:6, p 216-250).

In some embodiments, the pellet or mesh comprises one or more additional ingredients e.g., to stabilize or preserve the activity of the chemotherapeutic, to modulate the dissolution or dispersal rate of the chemotherapeutic, to maintain sterility of the chemotherapeutic, etc. These ingredients include but are not limited to: antioxidants, bacteriostats, buffers, carbohydrates, chelating agents such as EDTA or glutathione, coloring, diluents, emulsifiers, excipients, flavoring and/or aromatic substances, lubricants, pH buffering agents, physiologically acceptable carriers, polypeptides (e.g., glycine), preservatives, proteins, salts for influencing osmotic pressure, solubilizers, stabilizers, surfactants, wetting agents, etc. Buffers include but are not limited to saline, neutral buffered saline, phosphate buffered saline, etc. Carbohydrates include but are not limited to dextrans, glucose, mannose, mannitol, sucrose, etc. Stabilizing excipients include but are not limited to sugars, carbohydrates, lipids and various polymers.

In certain embodiments, the tumor or cancer arises from a hyperproliferative disorder or condition. In some cases, the cancer is a solid tumor. Examples of solid tumors include malignancies, e.g., adenocarcinomas, carcinomas, and sarcomas of the various organ systems, such as those affecting the bladder, brain, breast, cervix, colorectal, digestive/gastrointestinal system, gallbladder, head and neck, intestines, kidney, liver, lymphoid, lung, muscle ovarian, pancreas, pharynx, prostate, rectum, renal system, skin, soft tissue, thyroid, uterus, urothelial, and vagina. The cancer may be at an early, intermediate, late stage, metastatic cancer, etc.

Exemplary chemotherapeutic or cytotoxic agents that can be administered in pellet form, dispersed through the mesh, or in a dual pellet including both the chemotherapeutic agent and the radioactive element, include but are not limited to: alkylating agents; antitumor antibiotics, anthracyclines, anti-metabolites, mitotic inhibitors, plant alkaloids, topoisomerase inhibitors, or other miscellaneous antineoplastic agents.

Alkylating agents include but are not limited to: mustard gas derivatives (e.g., mechlorethamine, cyclophosphamide, chlorambucil, melphalan, and ifosfamide), ethylenimines (e.g., thiotepa and hexamethylmelamine); alkylsulfonates (e.g., busulfan), hydrazines and triazines (e.g., altretamine, procarbazine, dacarbazine and temozolomide); nitrosureas (e.g., carmustine, lomustine, and streptozocin); and metal salts (e.g., carboplatin, cisplatin, cis-dichlorodiamine platinum (II) cisplatin, and oxaliplatin) as well as melphalan, cyclothosphamide, dibromomannitol, streptozotocin, mitomycin C.

Anti-metabolites include but are not limited to: folic acid antagonists (e.g., methotrexate), pyrimidine antagonists (e.g., 5-fluorouracil, 5-fluorouracil decarbazine, foxuridine, cytarabine, capecitabine, and gemcitabine), purine antagonists (e.g., 6-mercaptopurine and 6-thioguanine), adenosine deaminase inhibitor (e.g., cladribine, fludarabine, nelarabine and pentostatin) as well as 6-mercaptopurine and 6-thioguanine.

Antitumor antibiotics, include but are not limited to: anthracyclines (e.g., doxorubicin, daunorubicin, epirubicin, mitoxantrone, and idarubicin), chromomycins (e.g., dactinomycin and plicamycin), mitomycin or bleomycin.

Mitotic inhibitors include but are not limited to: vincristine, vinblastine, taxol and maytansinoids.

Plant alkaloids, include but are not limited to: vinca alkaloids (e.g., vinblastine, vincristine, and vinorelbine); taxanes (e.g., paclitaxel and docetaxel); podophyllotoxins (e.g., etoposide and tenisopide), camptothecan analogs (e.g., irinotecan and topotecan).

Topoisomerase inhibitors include but are not limited to: topoisomerase I inhibitors (e.g., irinotecan or topotecan); or topoisomerase II inhibitors (e.g., amsacrine, etoposide, etoposide phosphate, teniposide).

Miscellaneous antineoplastics include but are not limited to: ribonucleotide reductase inhibitors (e.g., hydroxyurea); adrenocortical steroid inhibitors (e.g., mitotane); enzymes (e.g., asparaginase or pegaspargase), antimicrotubule agents (e.g., estramustine), retinoids (e.g., such as bexarotene, isotretinoin, tretinoin (ATRA)).

Additional chemotherapeutic agents include compounds that are capable of interfering with signal transduction pathways, agents that promote apoptosis, or proteosome inhibitors.

Radioactive include α-, β-, or γ-emitters, or β- and γ-emitters. Such radioactive isotopes include, but are not limited to: actinium (²²⁵Ac), astatine (²¹¹At), bismuth (²¹³Bi), carbon (¹⁴C), chromium (⁵¹Cr), chlorine (³⁶Cl), cobalt (⁵⁷Co or ⁵⁸Co), gallium (⁶⁷Ga), lutetium (¹⁷⁷Lu), indium (¹¹¹In), iodine (¹³¹I or ¹²⁵I), iron (⁵⁹Fe), yttrium (⁹⁰Y), phosphorus (³²P), praseodymium (¹⁴³Pr), rhenium (¹⁸⁶Re), rhodium (¹⁸⁸Rh), selenium (⁷⁵Se), sulfur (³⁵S), technetium (⁹⁹Tc), tritium (³H), etc.

The pellets or meshes can be prepared with a pharmaceutically acceptable carrier, which can be, for example, any suitable pharmaceutical excipient. The carrier includes any and all binders, fillers, solvents, dispersion media, coatings, surfactants, antioxidants, preservatives (e.g., antibacterial agents, antifungal agents), isotonic agents, absorption delaying agents, salts, drug stabilizers, disintegration agents, lubricants, sweetening agents, flavoring agents, dyes, and the like and combinations thereof, as would be known to those skilled in the art (see, for example, Remington's Pharmaceutical Sciences, 18th Ed. Mack Printing Company, 1990, pp. 1289-1329; Remington: The Science and Practice of Pharmacy, 21st Ed. Pharmaceutical Press 2011 and subsequent versions thereof). Except insofar as any conventional carrier is incompatible with the active ingredient, its use in the therapeutic or pharmaceutical compositions is contemplated. Other disclosure herein relating to the pellets or meshes can also be followed.

In some embodiments, the biodegradable pellets comprise a polymer that is dissolvable, or insoluble but dispersible under physiological conditions, or a combination of both. Polymers may include (e.g., polymers such as PLGA, carboxymethylcellulose (CMC), hyaluronic acid etc.). The pellets may further comprise micro- or nano-particles comprising absorbed, conjugated, dispersed, or encapsulated therapeutics, wherein the micro- or nano-particles are designed to deliver the therapeutic to the subject. Many materials suitable for forming the polymer are known within the art, and all such materials are contemplated for use herein.

In terms of arrangement of the multilayered mesh, in some embodiments, chemotherapy elements may be placed in the inner layer to facilitate “leaching out” of the therapeutic agent once the radiation dose has been delivered resulting in sequential anti neoplastic therapy. Radioactive elements may be placed in the outer layers, as these elements can penetrate through layers.

When designing a mesh, properties of meshes, e.g., the type of filament, the tensile strength, and the porosity, should all be considered. These properties determine the weight of the mesh and its biocompatibility. Meshes should be designed to have a sufficient tensile strength to be able to perform their function, e.g., in terms of providing support and isolating spaces or compartments, e.g., the pelvis, delivering therapeutic agents, etc. In preferred embodiments, light-weight meshes are selected due to their increased flexibility and reduction in complications. Larger pores correlate with increased rates of incorporation and decreased risk of encapsulation. For meshes placed in the peritoneal cavity, consideration should also be given to the risk of adhesion formation. Many different configurations are possible, and all such configurations fall within the scope of present invention embodiments.

The subject or recipient may be mammalian, and in particular, a human. In other embodiments, the subject or recipient may include domestic or livestock animals, e.g., cats, cows, dogs, goats, guinea pigs, horses, pigs, rabbits, rodents, or sheep.

The subject or recipient may be a human individual, suffering from cancer. The subject or recipient may be a child or an adult. The subject or recipient may be a healthy individual, at risk of developing cancer, e.g., a human undergoing a mastectomy for prevention of breast cancer might also benefit from the multilayer meshes described herein.

It should be apparent to those skilled in the art that many more modifications besides those already described are possible without departing from the inventive concepts herein. The inventive subject matter, therefore, is not to be restricted except in the scope of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification claims refers to at least one of something selected from the group consisting of A, B. C . . . and N, the text should be interpreted as requiring only one element from the group, not A plus N, or B plus N, etc.

The Examples provided herein are meant to assist one of ordinary skill in the art in carrying out the invention and are not intended in any way to limit the scope of the invention.

### EXAMPLES

### Example 1. Fabrication of Meshes

The meshes disclosed herein may be formed or derived from human, porcine or other biosynthetic materials. In some embodiments, meshes are obtained from dermal tissue, bladder tissue, intestinal tissue, or any other suitable tissue or organ comprising extracellular matrix or connective tissue. In some embodiments, a multi-layer mesh formed of human or porcine tissue or organs are formed from individual layers, with each layer having a chemotherapeutic agent or a radioactive element or a combination of both therein, and wherein the individual layers undergo bonding or high compression to join together and form a mesh suitable for implantation.

In still other embodiments, 3-D printing may be used to print a mesh of biological or biosynthetic materials with embedded chemotherapeutics and other biologic factors. The mesh may be printed in multiple layers, with a given layer having the same composition or having a different composition (e.g., with respect to chemotherapeutic agents, radioactive agents, or a combination of chemotherapeutic and radioactive elements).

As a non-limiting example, in some embodiments, a human or porcine dermis, intestine, bladder, or any suitable source obtained from a mammal, may undergo a decellularization process to remove cells, leaving behind a mesh formed primarily of extracellular matrix, e.g., a scaffold of collagen and elastin and optionally with growth factors. See, e.g., U.S. Pat. No. 6,893,666. In some embodiments, the mesh will also have pores. Chemotherapeutic agents may be incorporated into the layers of mesh using any suitable technique, including coating the chemotherapeutic agent on the surface of the mesh layer, soaking the mesh in a solution comprising one or more chemotherapeutic agents, or forming pellets comprising chemotherapeutic agents that are embedded in the mesh.

Similarly, for layers involving radioactive elements, the radioactive agent may be applied as a coating on the surface of the mesh layer, by soaking the mesh in a solution comprising one or more radioactive elements, or by forming biodegradable pellets comprising radioactive agents that are embedded in the mesh. For layers that involve combinations of chemotherapeutic and radioactive elements, such combinations may be applied in a similar manner.

Techniques for forming biological meshes from mammalian tissues are known in the art, see, e.g., FitzGerald et al., Biologic versus Synthetic Mesh Reinforcement: What are the Pros and Cons? Clin Colon Rectal Surg (2014) 27:140-148.

In another non-limiting example, layers of the mesh may be synthesized or grown, in vitro. Fibroblasts or other extracellular matrix secreting cells may be cultured to produce an extracellular matrix, according to available techniques, see, e.g., Scherzer et al., “Fibroblast-Derived Extracellular Matrices: An Alternative Cell Culture System That Increases Metastatic Cellular Properties” PLOS (2015) 10(9):e0138065.

In another non-limiting example, each layer of the mesh may be generated by 3D printing with a biological material. Techniques for printing biological materials are known in the art, e.g., see, Murphy et al., “3D bio-printing of tissues and organs” Nature Biotechnology (2013) 32:773-785. 3D printing allows meshes to be printed in an additive manner such that layers with differing compositions can be printed on top of each other, e.g., an inner layer may be printed with a chemotherapeutic agent, an outer layer may be printed with a radioactive agent, and so forth. A given layer having chemotherapeutic and/or radioactive elements may be additively printed on another layer having chemotherapeutic and/or radioactive elements.

Of course, a layer may be printed with a 3D process, the layer may be embedded with chemotherapeutic and/or radioactive pellets, coated with chemotherapeutic and/or radioactive elements, etc., and a subsequent layer of the mesh may then be printed on the modified layer. Many such modifications are understood to be within the scope of the embodiments disclosed herein.

### Example 2. Fabrication of Pellets

According to the embodiments disclosed herein, radioactive or chemotherapeutic agents may be incorporated into dissolvable pellets, which are then embedded into the mesh. Techniques for forming pellets may be based on e.g., U.S. Pat. No. 6,248,057. Briefly, bioabsorbable materials, such as polymers, may be mixed with the chemotherapeutic agent or radioactive element, and formed into a pellet.

### Example 3. Surgical Implantation of Meshes or Pellets

As described herein, the multilayer biological meshes are suitable for surgical implantation. A patient who has been diagnosed with cancer may undergo surgery to remove part or all of the cancer. As part of the procedure, the surgeon resects part or all of the tumor and places the mesh at the site where the tumor was removed in the tumor bed. In still other embodiments, for sites where the tumor is unresectable or is too close to vascular structures for a safe excision, the surgeon may embed the mesh therein in order to shrink or otherwise impede the growth of the tumor.

In some embodiments, the surgical meshes are designed to degrade slowly, over a period of weeks, months, or years. In other embodiments, the meshes are designed to degrade rapidly, within a matter of hours or days. By designing the mesh to have varying degradation rates, delivery of one or more therapeutics can be customized to a particular patient and type of tumor/cancer.

The thickness of the mesh may be based on the time frame in which the therapy is needed. The size of the mesh can be customized to fit core (body) sizes. During surgical placement, meshes can be cut down to size, e.g., to fit into a particular location, or multiple meshes may be placed together for larger target sites. To prevent migration after surgical placement, the mesh may be sutured or glued into place.

The implantable meshes, as described herein, have a number of advantages over existing meshes. For example, the multilayer biological meshes can deliver both radioactive elements and chemotherapeutic agents to a desired site, e.g., a tumor bed, within a solid tumor, etc. In some embodiments, the mesh may be bioabsorbed over a relatively long timeframe, so that the mesh remains in a fixed or relatively fixed position, allowing the continuous delivery of chemotherapeutic and radioactive elements to a particular site. Thus, these meshes are bioabsorbable, and as the absorption process continues, so does the degradation of the mesh, along with continuous release of chemotherapeutic agents and radioactive elements. Thus, unlike delivery using pellets, which can translocate to a different position or even a completely different position in the body, the meshes herein are fixed until they are absorbed.

In some embodiments, the layers of the mesh may be configured to have different bioabsorption rates. For example, an outer layer could be designed, e.g., based on the type of polymer, based on the thickness and the porosity of the layer, etc., to bioabsorb rapidly—thus, rapidly delivering a high dose of one or more therapeutics to the target site. The inner layers can be designed to have different properties, having a bioabsorption rate of a slower timeframe, thus delivering a steady dose of the therapeutic agent on a long time frame to kill any residual tumor cells. These approaches are anticipated to lead to lower levels of cancer recurrence and resistance, as residual cells (not killed by an initial treatment) will be targeted and killed over a longer timeframe.

These examples are purely intended to be exemplary and are not intended to be limiting, as numerous different embodiments are understood to fall within the scope of present invention embodiments.

What is claimed is:

1. A surgical multilayer mesh for the treatment of cancer comprising:
a bioabsorbable material;
a chemotherapeutic agent; and
a radioactive element.

2. The surgical mesh of claim 1, wherein the surgical mesh comprises:
one or more inner or outer layers comprising the radioactive element; and
one or more inner or outer layers comprising the chemotherapeutic agent.

3. The surgical mesh of claim 1, comprising:
two or more layers comprising the radioactive element and the chemotherapeutic agent in the same layer.

4. The surgical mesh of claim 1, wherein the surgical mesh comprises a pore size ranging from 50 um to 5 mm.

5. The surgical mesh of claim 1, wherein the radioactive element is provided in the form of a pellet.

6. The surgical mesh of claim 1, wherein the chemotherapeutic agent is provided in the form of a pellet.

7. The surgical mesh of claim 2, wherein the radioactive element is provided in the form of a pellet embedded in the one or more inner layers.

8. The surgical mesh of claim 2, wherein the chemotherapeutic agent is provided in the form of a pellet embedded in the one or more outer layers.

9. The surgical mesh of claim 2, wherein the radioactive element is provided in the form of a pellet embedded in the one or more inner layers, and the chemotherapeutic agent is provided in the form of a pellet embedded in the one or more outer layers.

10. The surgical mesh of claim 1, wherein the radioactive element and the chemotherapeutic agent together are provided in the form of a pellet, wherein the pellet is embedded in the surgical mesh, and wherein the radioactive element and the chemotherapeutic agent are dispersed throughout a same layer of the pellet.

11. The surgical mesh of claim 1, wherein the radioactive element and the chemotherapeutic agent together are provided in the form of a pellet, wherein the pellet is embedded in the surgical mesh, and wherein the radioactive element is dispersed throughout an inner layer of the pellet, and the chemotherapeutic agent is dispersed throughout an outer layer of the pellet.

12. The surgical mesh of claim 1, wherein the mesh comprises a biodegradable biosynthetic material.

13. The surgical mesh of claim 1, wherein the mesh comprises a material derived from a human.

14. The surgical mesh of claim 1, wherein the mesh comprises a material that is porcine urinary bladder.

15. The surgical mesh of claim 1, wherein the mesh biodegrades when exposed to physiological conditions.

16. The surgical mesh of claim 15, wherein the physiological conditions comprise one or more of:
(a) an aqueous environment; or
(b) a temperature of 21° C. to 37° C.

17. A radioactive pellet for the treatment of cancer, wherein the pellet is fully biodegradable and comprises an inner layer comprising one or more radioactive elements.

18. The radioactive pellet of claim 17, wherein the inner layer additionally comprises one or more chemotherapeutic agents.

19. A method of treating a patient comprising implanting the surgical multilayer mesh of claim 1 within the patient.

20. A method of manufacturing a surgical multilayer mesh or a biodegradable pellet comprising:
a bioabsorbable material;
a chemotherapeutic agent; and
a radioactive element.
